(19) United States
(12) Patent Application Publication
(10) Pub. No.: US 2021/0293817 A1
(43) Pub. Date: Sep. 23, 2021

(54) METHOD AND DEVICE FOR DETECTIONING AND MONITORING THE PRESENCE, DEVELOPMENT AND PROPAGATION OF INFECTIOUS AGENTS, IN PARTICULAR BACTERIA AND VIRUSES

(71) Applicants: Morou BOUKARI, Toulouse (FR); PRODOSE, Bessieres (FR)

(72) Inventor: Morou BOUKARI, Toulouse (FR)

(21) Appl. No.: 17/247,892

(22) Filed: Dec. 29, 2020

Related U.S. Application Data
(63) Continuation of application No. PCT/FR2020/000085, filed on Apr. 3, 2020.
(60) Provisional application No. 62/992,309, filed on Mar. 20, 2020.
(60) Provisional application No. 62/994,048, filed on Mar. 24, 2020.

(51) Int. Cl. G01N 33/569; C12Q 1/689; C12Q 1/70; G01N 1/24; G01N 1/34

(52) U.S. Cl. CPC G01N 33/56983; G01N 33/56911; G01N 1/34; C12Q 1/70; G01N 1/24; C12Q 1/689

(57) ABSTRACT

Disclosed is a method for detecting and monitoring the presence, development and propagation of at least one infectious agent such as a bacterium or a virus, which is remarkable in that it includes the following steps: Subdivide the considered geographic area into area cells; Determine and identify in each area cell the points of presence, passing through or frequentation by human and/or animal beings likely to be infected by the infectious agent or by human and/or animal beings likely to be hosts to the infectious agent; and Install at each identified point in each area cell an autonomous device for detection of the infectious agent present in the environment surrounding the device by analysis and identification. Also disclosed is a device for executing the process.

### FIELD OF APPLICATION OF THE INVENTION

The invention relates to the field of the detection of infectious agents such as bacteria and viruses present in an environment, and in particular to the adaptations making it possible to implement this detection under the best conditions.

### DESCRIPTION OF PRIOR ART

There are a number of tests for detecting and identifying a bacterium or a virus. However, in most cases, these tests on patients are performed after the epidemic is observed (these patients may be subject to surveillance).

In addition, once the epidemic has been observed, the detection of its presence and its follow-up depend on monitoring new patients or not.

Once the epidemic has been observed, there are means of remote temperature measurement of patients, who can be geo-located and tracked by their telephone to allow the health authorities to treat him and prevent the rest of the population from being in contact with him. This type of monitoring can pose a problem for its legal application. In addition, it does not allow the presence of the virus to be monitored in the absence of a detected patient. Thus, a healthy carrier or one who does not have the searched symptom (temperature here) or the absence of a patient (due to a containment measure) are all cases that do not allow proper monitoring of the epidemic.

Furthermore, it does not solve the problem of detecting and tracking the bacteria or virus before the epidemic occurs or at the very beginning of this epidemic.

### BRIEF DESCRIPTION OF THE INVENTION

Based on the foregoing, the applicants carried out research aimed at:
detecting the presence, development and propagation of infectious agents such as viruses and/or bacteria present in airborne liquid microdroplets (emitted by human and/or animal beings infected with viruses and/or bacteria or by human and/or animal beings which host these viruses and/or bacteria) at a considered point in a considered geographical area,
monitoring the propagation of viruses and/or bacteria present in airborne microdroplets of liquid at different considered points in a considered geographical area,
avoiding conducting infection tests on every individual of the human and/or animal beings,
detecting as early as possible the emergence of an infection with the virus and/or bacteria responsible for epidemics and pandemics.

This research has led to a process for detecting and monitoring the presence, development and propagation of at least one infectious agent such as a bacterium or a virus, remarkable in that it includes the following steps:
Subdivide the considered geographical area into area cells,
Determine and identify in each area cell the points of presence, passing through or frequentation by human and/or animal beings likely to be infected by the infectious agent or by human and/or animal beings likely to be hosts of that infectious agent,

Install at each identified point in each area cell an autonomous detection device for detection of the infectious agent present in the surrounding environment of the device by analysis and identification.

The invention is particularly advantageous in that it proposes to predispose in the territory devices, terminals, stations or probes for detecting infectious agents (bacteria and/or viruses) which are equipped with adequate detection, identification and analysis means. These means analyze the environment, the fluid at the identified points.

According to another particularly beneficial feature, said environment is air or water. When the environment, the fluid analyzed is water, the latter may be water from one of the following environments: River, lake, ocean, stream, sea, water table, rain, well, etc.

The identified patients are thus not monitored but the presence of the infectious agent in the air. The latter can therefore be detected before the epidemic is declared. In addition, it can be detected and monitored despite the presence of asymptomatic patients or despite the absence of a patient to monitor due to a containment measure.

The invention also has the great advantage of not arising a legal problem as regards to its implementation.

According to another particularly advantageous feature of the invention, the method is applicable to an infectious agent which is indifferently:
alone, suspended in the air,
adsorbed on airborne solid particles,
adsorbed on airborne chemical molecules,
present in chemical molecules emitted into the air by human and/or animal beings,
present in airborne liquid particles.

According to another particularly advantageous characteristic of the invention, the analysis and identification of the infectious agent are carried out by one or more of the following methods:
RT-PCR method,
Method of searching for antibodies specific to each virus and/or each bacterium,
Meta-genomic method (Immunoassay), i.e. a method of searching for nucleic acid specific to each virus and/or each bacterium.

According to another particularly advantageous feature of the invention, the method comprises a step of aspiration of the environment surrounding the autonomous device towards the interior of the device. The air surrounding the device is thus sucked in, when the device is installed on land.

According to another particularly advantageous feature of the invention, the aspiration of the environment surrounding the autonomous detection device takes place at fixed time intervals.

According to another particularly advantageous feature of the invention, the aspiration of the environment surrounding the autonomous detection device takes place at variable time intervals and depending on the frequency of visit of each point of installation of the device.

According to a particularly advantageous feature of the invention, the method consists in analyzing the airborne microdroplets at the identified points, emitted by human and/or animal beings likely to be infected by the infectious agent or by human and/or animal beings likely to be hosts of said infectious agent.

According to another particularly advantageous feature of the invention, the method applies to at least one of the following infectious agents and the device for carrying out the method is equipped for the detection of at least one of the following infectious agents:
Ebola virus.
virus responsible for COVID-19.
virus responsible for any epidemic and/or pandemic.
bacteria responsible for any epidemic and/or pandemic.

The invention also concerns the device for implementing the above described method, which autonomous device is remarkable in that it includes:
Air extraction means,
Means and reagents for extracting the infectious agent or the genome of the infectious agent or specific substances and proteins of the infectious agent extracted from the air,
Means for transmission by wireless link or by wire link of the results of extraction of the infectious agent.

According to another feature of the invention, the device only comprises the extraction means, the analysis and identification of the infectious agent being carried out afterwards in a specialized laboratory.

According to another feature of the invention, the device comprises means for analyzing and identifying the infectious agent extracted.

According to another feature of the invention, the device comprises means for recording and storing the results of analysis and identification of viruses and/or bacteria.

According to another particularly advantageous feature of the invention, the extraction of the air is carried out by suction of the air surrounding the autonomous detection device using an air pump, which suction of air is followed by filtration of the air.

According to another particularly advantageous feature of the invention, the filtration of the air is carried out by at least one of the following means:
filtration of the sucked air on an absorbent membrane,
filtration of the sucked air on a membrane absorbing the microdroplets present in the air,
filtration of the sucked air on a repellent membrane,
filtration on any type of membrane,
filtration of the sucked air on a membrane repelling microdroplets suspended in the sucked air and followed by collection in liquid form of these microdroplets.

According to another particularly advantageous feature of the invention, the membrane is soluble in the reagent(s) used to isolate or purify the infectious agent(s) or their genome or characteristic substances or proteins.

According to another particularly advantageous feature of the invention, the extraction of the microdroplets by air suction is followed by condensation and subsequent collection of the condensates.

According to another particularly advantageous feature of the invention, this condensation is carried out by means of cryogenic cooling of the sucked air, implemented by an electric cooling module.

According to another feature of the invention, the device includes a sensor for detecting the passage or presence of human or animal beings, making it possible to trigger the suction.

According to another feature of the invention, the autonomous device comprises one or more disinfection means selected from the following list:
physical means of heat generation;
physical means of UV generation;
chemical reagents for disinfecting, between each analysis, the circuit for aspiration and identification of the infectious agent.

According to another particularly advantageous feature of the invention, the autonomous device includes reagents that neutralize the disinfecting reagents between each analysis.

According to another feature of the invention, in order to increase the operating autonomy of the device and also to avoid contamination of the various analytical samples, the autonomous device comprises a plurality of independent cartridges for the extraction of infectious agent, which cartridges are mounted in parallel with isolation means for isolating each cartridge upstream and downstream and with a single cartridge or a limited number of cartridges that can operate simultaneously.

According to an embodiment, all the cartridges are connected, upstream and ahead of the isolation means of each cartridge, to the outlet of a single pump for the aspiration of the microdroplets or aerosols containing infectious agents.

In one variant, all the cartridges are connected, downstream and after the isolation means of each cartridge, to the inlet of a single pump for the aspiration of the air containing microdroplets or aerosols likely to contain infectious agents.

According to another feature of the invention, in order to increase the operating autonomy of the device and also to avoid contamination of the different analytical samples, the autonomous device comprises several independent circuits mounted in parallel and each comprising means for extracting infectious agent from viruses and/or bacteria and means of analyzing and identifying the infectious agent, which circuits are mounted in parallel with means for isolating each circuit upstream and downstream.

According to another particularly advantageous feature of the invention, the autonomous device is supplied with electrical power via one or more of the following supply means:
public power grid,
autonomous battery,
solar panel,
wind turbine.

According to another particularly advantageous feature of the invention, the autonomous device is installed at a height in relation to the ground between 0 m (on the ground) and 2.5 m (maximum height of the human being).

According to another feature of the invention, the device includes geolocation means.

According to another particularly advantageous feature of the invention, the device comprises an enclosure containing all or part of the means described above and closed by walls in which orifices are arranged, which orifices allow inside passage of the surrounding air by suction.

According to another particularly advantageous feature of the invention, the follow-up of the propagation of at least one infectious agent present in the air at different considered points of a considered geographical area are carried out by a process of reverse location search. That is, a user of the process can know, without knowing the name of the place, where and in what quantity an infectious agent is detected.

Which reverse search procedure is characterized by the fact that it associates:
artificial intelligence present in a terminal (smartphones, PCs, tablets, vehicle on-board computers) to analyze, interpret and process locally the desires and wishes clearly expressed by the user,
Instantaneous data from devices, which autonomous devices are distributed over the earth's surface and inform about the presence of the infectious agent in every place on the globe,
data from other sources associated with each location on the globe,
one or more servers connected by digital links (satellite, 5G, 4G, LTE, cable, fiber optics, low-speed networks, high-speed networks, IoT networks, etc.) to terminals and autonomous devices.

Thus, a user searching for a location can define as search parameters medical criteria or the presence of one or more infectious agents. The process enables him/her, thanks to autonomous devices, to provide data that meet this search parameter.

Since the fundamental concepts of the invention have just been set out above in their most elementary form, other details and characteristics will emerge more clearly on reading the description that follows and in relation to the appended drawings, supplying, as non-limiting examples, a mode of execution of a device according to the invention.

### BRIEF DESCRIPTION OF THE DRAWINGS

FIG. 1 is a schematic drawing of an embodiment of an autonomous device for the detection and identification of viruses and bacteria according to the invention;

FIG. 2 is a schematic drawing of the device of FIG. 1 installed in nature;

FIG. 3 is a schematic drawing of the device of FIG. 1 installed in an urban environment.

### DESCRIPTION OF AN EMBODIMENT

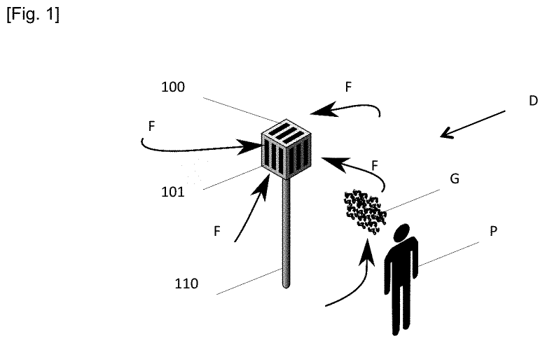

As shown in FIG. 1, the device, in the form of an autonomous terminal for the detection and identification of viruses and/or bacteria, is denoted D as a whole. It consists of a cubic-shaped housing 100 with slots 101 on the sidewalls. This 100 housing is mounted at the end of a pole 110.

Various means of suction, detection, etc. . . . of viruses and/or bacteria in device D are arranged in the housing 100. The suction means draw air surrounding the device D (arrows F) and in particular the droplets G emitted by the person P. Once viruses or bacteria detected, the device D transmits the information.

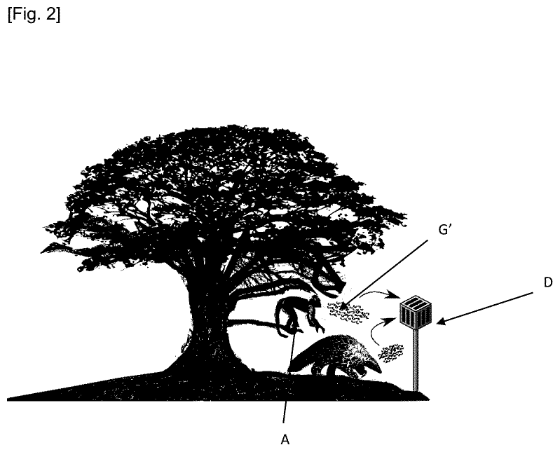

FIG. 2 illustrates a situation where device D is installed in a forest environment and sucks droplets G′ (or aerosols) emitted by animals A regularly or once detected.

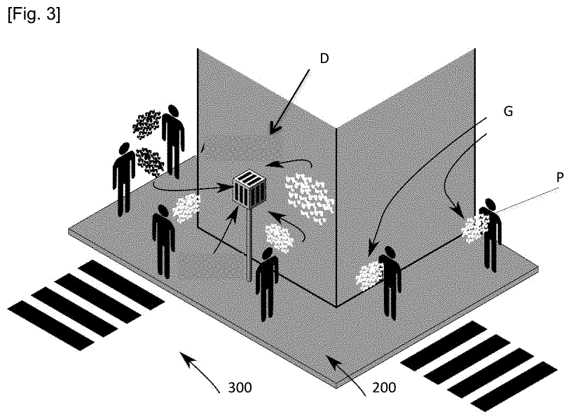

FIG. 3 illustrates a situation where device D is installed in an urban environment, i.e., here on a sidewalk 200 near roads 300. The device D draws air that surrounds its location and thus the droplets G emitted by persons P walking nearby, or those present on inert supports nearby.

It is understandable that the devices described above and represented above were intended for disclosure rather than limitation. Of course, various adjustments, modifications and improvements can be made to the above examples, without going beyond the scope of the invention.

1. A method for detecting and monitoring the presence, development and propagation of at least one infectious agent the method comprising:
Subdivide the considered geographic area into area cells,
Determine and identify in each area cell the points of presence, passing through or frequentation by human and/or animal beings likely to be infected by the infectious agent or by human and/or animal beings likely to be hosts of said infectious agent,
Install at each identified point in each area cell an autonomous detection device (D) for detection of the infectious agent present in the surrounding environment of the device (D) by analysis and identification.

2. The method according to claim 1, wherein said environment is air or water.

3. The method according to claim 1, wherein the method applies to an infectious agent which is indifferently:
alone, suspended in the air,
adsorbed on airborne solid particles,
adsorbed to airborne chemical molecules,
present in chemical molecules emitted into the air by human and/or animal beings,
present in airborne liquid particles.

4. The method according to claim 1, wherein the analysis and identification of the infectious agent are carried out by one or more of the following methods:
RT-PCR method,
Method of searching for specific antibodies to each virus and/or bacterium,
Metagenomic method (Immunoassay), i.e. a method of searching for specific nucleic acid of each virus and/or bacterium.

5. The method according to claim 1, further comprising it a step of aspiration of the environment surrounding the autonomous device (D) towards the interior of the device (D).

6. The method according to claim 5, wherein the aspiration of the environment surrounding the autonomous detection device (D) takes place at fixed time intervals.

7. The method according to claim 5, wherein the aspiration of the environment surrounding the autonomous detection device (D) takes place at varying time intervals and depending on the frequency of visit of each point of installation of the device (D).

8. The method according to claim 1, wherein the method is applied to analyzing the airborne microdroplets at the identified points, emitted by human and/or animal beings likely to be infected by the infectious agent or by human and/or animal beings likely to be hosts of said infectious agent.

9. The method according to claim 1, wherein the method is applied to at least one of the following infectious agents, and the device (D) for carrying out this method is equipped for the detection of at least one of the following infectious agents:
Ebola virus.
virus responsible for COVID-19.
virus responsible for any epidemic and/or pandemic.
bacteria responsible for any epidemic and/or pandemic.

10. A device (D) for implementing the method according to claim 1, comprising:
Environment extracting means,
Means and reagents for extracting the infectious agent or the genome of the infectious agent or specific substances and proteins of the infectious agent extracted from the environment,
Means for transmission by wireless link or by wire link of the results of extraction of the infectious agent.

11. The device (D) according to claim 10, further comprising means for analyzing and identifying the infectious agent extracted.

12. The device (D) according to claim 10, further comprising means for recording and storing the results of analysis and identification of viruses and/or bacteria.

13. The device (D) according to claim 10, wherein the extraction of the air is carried out by suction of the air surrounding the autonomous detection device (D) using an air pump, which suction of air is followed by filtration of the air.

14. The device (D) according to claim 13, wherein the filtration of the air is carried out by at least one of the following means:
filtration of the sucked air on an absorbent membrane,
filtration of the sucked air on a membrane absorbing the microdroplets present in the air,
filtration of the sucked air on a repellent membrane,
filtration of the sucked air on a membrane repelling microdroplets suspended in the sucked air and followed by collection in liquid form of these microdroplets.

15. The device (D) according to claim 14, wherein the membrane is soluble in the reagent(s) used to isolate or purify the infectious agent(s) or their genome or characteristic substances or proteins.

16. The device (D) according to claim 14, wherein the extraction of the microdroplets by air suction is followed by condensation and subsequent collection of the condensates.

17. The device (D) according to claim 16, wherein the condensation is carried out by means of cryogenic cooling of the sucked air, implemented by an electrical cooling module.

18. The device (D) according to claim 10, further comprising a sensor for detecting the passage or presence of human or animal beings, the sensor being arranged to trigger the suction when the passage is detected.

19. The device (D) according to claim 10, further comprising one or more disinfection means selected from the following list:
physical means of heat generation;
physical means of UV generation
chemical reagents for disinfecting, between each analysis, the circuit for aspiration and identification of the infectious agent.

20. The device (D) according to claim 10, further comprising reagents that neutralize the disinfecting reagents between each analysis.

21. The device (D) according to claim 10, further comprising a plurality of independent cartridges for the extraction of infectious agent, which cartridges are mounted in parallel with isolation means for isolating each cartridge upstream and downstream and with a single cartridge or a limited number of cartridges that can operate simultaneously.

22. The device (D) according to claim 21, wherein all the cartridges are connected, upstream and ahead of the isolation means of each cartridge, to the outlet of a single pump for the aspiration of the microdroplets or aerosols likely to contain infectious agents.

23. The device (D) according to claim 21, wherein all the cartridges are connected, downstream and after the isolation means of each cartridge, to the inlet of a single pump for the aspiration of the microdroplets or aerosols containing bacteria and/or viruses.

24. The device (D) according to claim 10, further comprising several independent circuits connected in parallel and each comprising means for extracting an infectious agent and means for analyzing and identifying the infectious agent, which circuits are connected in parallel with means for isolating each circuit upstream and downstream.

25. The device (D) according to claim 10, wherein the device is supplied with electrical power via one or more of the following means of supply:
public power grid,
autonomous battery,
solar panel,
wind turbine.

26. The device (D) according to claim 10, wherein the device is installed at a height in relation to the ground between 0 m (on the ground) and 2.5 m (maximum human height).

27. The device (D) according to claim 10, further comprising geolocation means.

28. The device (D) according to claim 11, further comprising an enclosure containing all or part of the means for analyzing and identifying the infectious agent extracted and closed by walls in which orifices are arranged, which orifices allow inside passage by suction of the surrounding air.
